(19) United States
(12) Patent Application Publication
(10) Pub. No.: US 2013/0072936 A1
(43) Pub. Date: Mar. 21, 2013

(54) ANGLED GRINDER

(71) Applicants: John To, Newark, CA (US); John Davis, Sunnyvale, CA (US); Singfatt Chin, Pleasanton, CA (US)

(72) Inventors: John To, Newark, CA (US); John Davis, Sunnyvale, CA (US); Singfatt Chin, Pleasanton, CA (US)

(21) Appl. No.: 13/509,279

(22) PCT Filed: Nov. 10, 2010
(86) PCT No.: PCT/US10/56246

§ 371 Date: Nov. 29, 2012

Related U.S. Application Data
(60) Provisional application No. 61/259,968, filed on Nov. 10, 2009.

(51) Int. Cl. A61B 17/16

(52) U.S. Cl. CPC A61B 17/16
USPC 606/79

(57) ABSTRACT

A tissue removal system comprises a rotatable burr element located within a distal housing and a burr opening. The burr opening is located about a distal surface and side surface of the distal housing. The distal housing is configured to bend or pivot with respect to a proximal housing. The proximal housing comprises an auger hole configured to draw fluid and particulate matter for transport proximally along the length of the tissue removal system. A linkage assembly attaches a drive shaft of the tissue removal assembly to the rotatable burr element to permit rotation of the rotatable burr element when the distal housing is bent or pivoted.

### CROSS-REFERENCE TO RELATED APPLICATIONS

This application claims benefit under 35 U.S.C. §119(e) to U.S. Provisional Application Ser. No. 61/259,968, filed on Nov. 10, 2009, which is hereby incorporated by reference in its entirety.

### BACKGROUND

Spinal stenosis is a disorder where narrowing occurs in the spaces of the spine. The disorder may affect the central canal of the spine in which the spinal cord is housed (e.g. central spinal stenosis) or the lateral foramina formed between two adjacent vertebrae from which the spinal nerves exit (e.g. lateral spinal stenosis). Spinal stenosis is frequently associated with degenerative disease of vertebral disc and/or vertebrae. The degenerative changes may cause reactive bony or ligament ingrowth and may reduce vertebral spacing, which may lead to nerve impingement. This nerve impingement may result in debilitating forms of sciatica, which is a radiating pain to limbs or upper body and further areas in the body, as well as limitations in physical movement due to this pain.

Temporary relief of pain of this condition is often sought through conservative therapy, which includes positional therapy (e.g. sitting or bending forward to reduce pressure on spine), physical therapy, and medication or drug therapy to reduce pain and inflammation. When conservative therapy fails to resolve a patient's symptoms, surgery may be considered to address the structural etiologies of the symptoms. Surgical treatments for suspected spinal stenosis often involve open procedures that require extensive dissection of muscle, connective tissue and bone along a patient's back to achieve adequate surgical exposure. These surgeries also expose the patient to a significant risk of complications, due to the presence of critical neurovascular structures near the surgical site. Specific surgical treatments include 1) foraminotomy, which involves the removal of bone surrounding an impinged nerve, 2) laminectomy, where the arch-like bone forming the posterior border of the spinal canal is removed to relieve pressure on the nerve roots or spinal cord, 3) discectomy, which involves removal of vertebral disc material impinging on a nerve, and 4) spinal fusion, which involves the use of grafts or implants to stabilize the movement between two vertebrae by eliminating any relative motion between them.

### BRIEF SUMMARY

In some examples, a tissue removal system comprises a rotatable burr element located within a distal housing and a burr opening. The burr opening is located about a distal surface and side surface of the distal housing. The distal housing is configured to bend or pivot with respect to a proximal housing. The proximal housing comprises an auger hole configured to draw fluid and particulate matter for transport proximally along the length of the tissue removal system. A linkage assembly attaches a drive shaft of the tissue removal assembly to the rotatable burr element to permit rotation of the rotatable burr element when the distal housing is bent or pivoted.

### BRIEF DESCRIPTION OF THE DRAWINGS

FIG. 1 is a schematic perspective view of a portion of a lumbar spine;

FIG. 2 is a schematic superior view of a portion of a lumbar vertebra and disc;

FIG. 3A is a schematic lateral view of a portion of a lumbar spine (without the spinal nerves); FIG. 3B depicts the portion of the lumbar spine in FIG. 3A (with the spinal nerves depicted);

FIGS. 4A and 4B are a side and rear elevational views of a burr system; FIG. 4C is a cutaway view of the burr system in FIG. 4A with a portion of the handle housing removed;

FIG. 5A is a perspective view of the distal end of the burr system in FIG. 4A; FIGS. 5B and 5C are side elevational views of the distal end of the burr system in a straight and a deflected position, respectively;

FIGS. 6A and 6B are longitudinal cross-sectional views of the distal end of the burr system, corresponding to FIGS. 5A and 5B, respectively; FIG. 6C is a longitudinal cross-sectional view of the distal end of the burr system, with the drive shaft rotated 90 degrees from the position illustrated in FIG. 6B;

FIGS. 7A and 7B are isolated views of the drive shaft, coupling joint and burr element without the burr housing and rotated 90 degrees apart;

FIGS. 8A and 8B depict the burr system of FIGS. 5B and 5C used with an exemplary endoscopic retractor system; and

FIGS. 9A to 9D depict an alternate configuration of the burr element.

### DETAILED DESCRIPTION

Medication and physical therapy may be considered temporary solutions for spine-related disorders. These therapies, however, may not fully address the underlying pathologies. In contrast, current surgical solutions such as laminectomy, where the laminae (thin bony plates covering the spinal canal) are removed, permit exposure and access to the nerve root which does address the underlying pathologies. From there, bone fragments impinging the nerves may be removed. Screws, interbody spacers, and fixation plates may also be used to fuse or stabilize the spine following laminectomy. These surgical techniques, however, are quite invasive and require extensive preparation and prolonged exposure time during the surgery, often prolonging an already significant recovery time. Removal of bone tissue in close proximity to nerves may also increase the risk of neurovascular damage. Other surgical methods have been attempted, such as laminotomy, which focuses on removing only certain portions or smaller segments of the laminae. Although removing less bone may be less invasive, risks of iatrogenic blood vessel and nerve damage may increase. Some spine procedures also utilize posterior approaches to the spine, which may require deliberate removal of an intervening spinous process merely to achieve access to the desired surgical site.

To be the least destructive to spine structures while preserving the strength of the bones, a spinal procedure may be minimally invasive while also reducing the amount of excised, native bone or dissection of surrounding native tissues. The exemplary embodiments described herein include but are not limited to minimally invasive access systems and methods for performing foraminotomy, and tools for removing bone while preserving the adjacent soft tissue such as nerves and blood vessels.

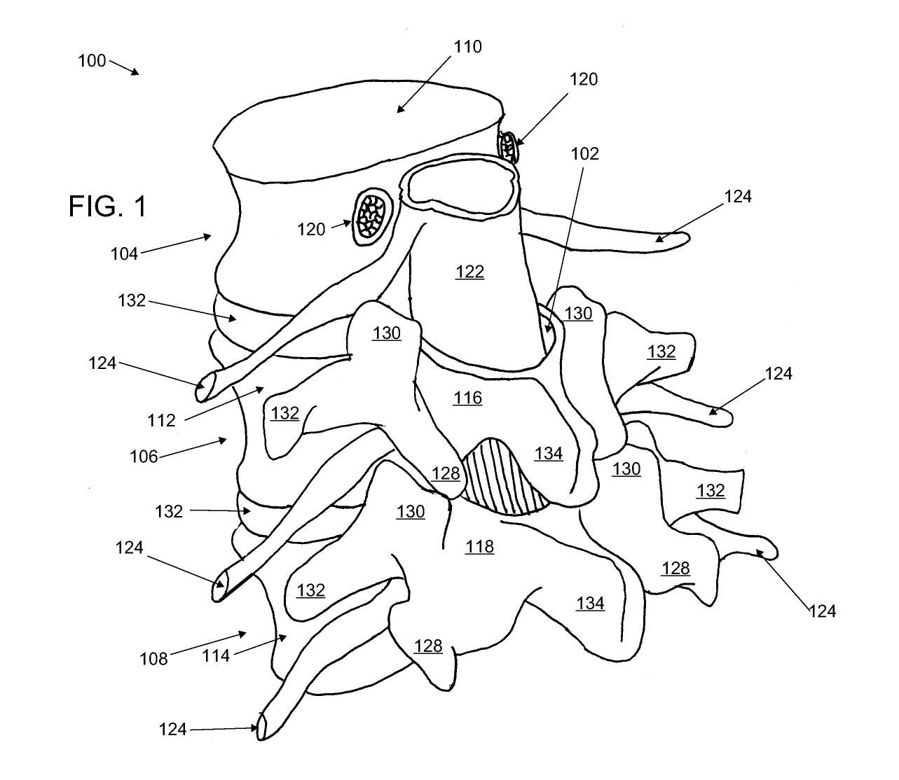

FIG. 1 is a schematic perspective view of a lumbar portion of a spine 100. The vertebral canal 102 is formed by a plurality of vertebrae 104, 106, and 108, which comprise vertebral bodies 110, 112, and 114 anteriorly and vertebral arches 116 and 118 posteriorly. The vertebral arch and adjacent connective tissue of the superior vertebra 104 in FIG. 1 has been omitted to better illustrate the spinal cord 122 within the vertebral canal 102. Spinal nerves 124 branch from the spinal cord 122 bilaterally and exit the vertebral canal 102 through intervertebral foramina 126 that are formed between adjacent vertebra 104, 106 and 108. The intervertebral foramina 126 are typically bordered by the inferior surface of the pedicles 120, a portion of the vertebral bodies 104, 106 and 108, the inferior articular processes 128, and the superior articular processes 130 of the adjacent vertebrae. Also projecting from the vertebral arches 116 and 118 are the transverse processes 132 and the posterior spinous processes 134 of the vertebrae 106 and 108. Located between the vertebral bodies 110, 112 and 114 are vertebral discs 132.

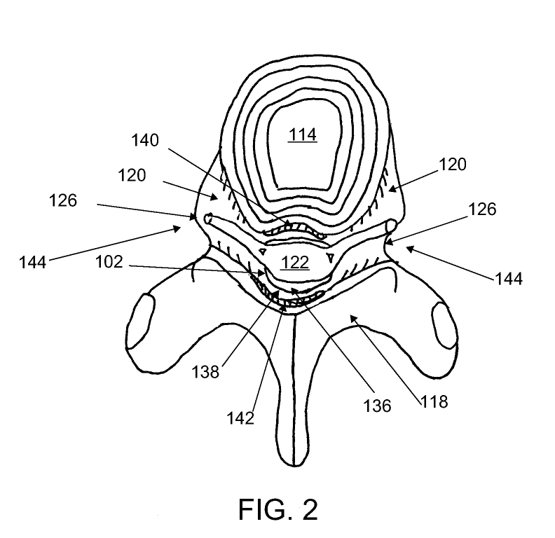

Referring to FIG. 2, the spinal cord 122 is covered by a thecal sac 136. The space between the thecal sac 136 and the borders of the vertebral canal 102 is known as the epidural space 138. The epidural space 138 is bound anteriorly and posteriorly by the longitudinal ligament 140 and the ligamentum flavum 142, respectively, of the vertebral canal 102, and laterally by the pedicles 120 of the vertebral arches 116 and 118 and the intervertebral foramina 126. The epidural space 138 is contiguous with the paravertebral space 144 via the intervertebral foramina 126.

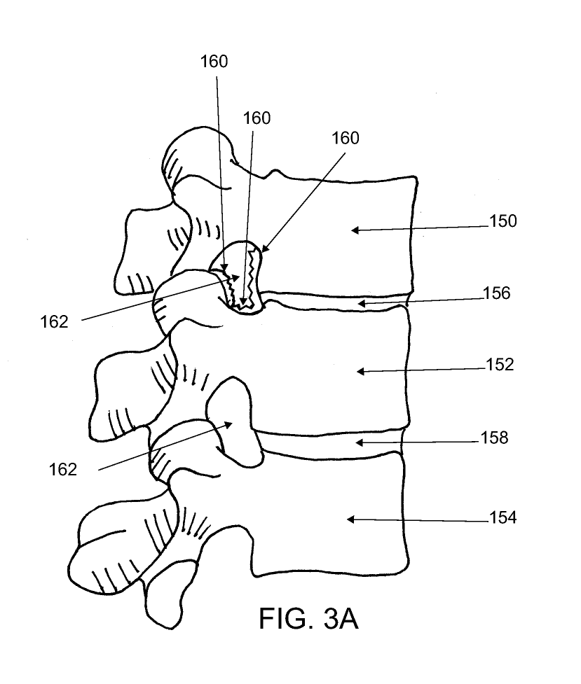

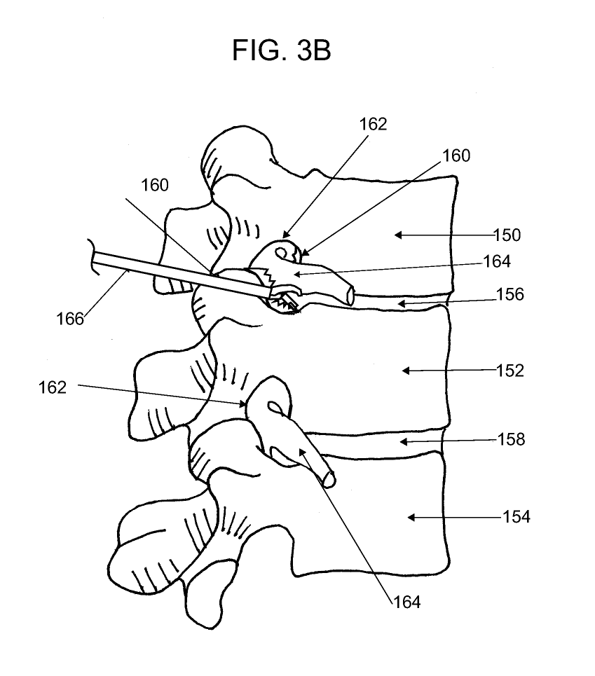

With degenerative changes of the spine, which include but are not limited to disc bulging and hypertrophy of the spinal ligaments and vertebrae, the vertebral canal 102 may narrow and cause impingement of the spinal cord or the cauda equina, a bundle nerves originating at the distal portion of the spinal cord. Disc bulging or bone spurs may also affect the spinal nerves 124 as they exit the intervertebral foramina 126. FIG. 3A, for example, schematically depicts a lateral view of three vertebrae 150, 152 and 154 with intervertebral discs 156 and 158, without the spinal cord or spinal nerves. With degenerative changes, regions of bone hypertrophy 160 may develop about the intervertebral foramina 162. While secondary inflammation of the associated nerve and/or soft tissue may benefit from conservative therapy, the underlying bone hypertrophy remains untreated. The regions of bone hypertrophy 160 may be removed, with or without other tissue, using open surgical spine procedures, limited access spine procedure, percutaneous or minimally invasive spine procedures, or combinations thereof. FIG. 3B depicts the vertebrae 150, 152 and 154 of FIG. 3A with their corresponding spinal nerves 164 during a foraminotomy procedure using a burr or grinder system 166. One example of a limited access spine procedure is disclosed in U.S. Pat. No. 7,108,705, which is hereby incorporated by reference in its entirety. Examples of percutaneous or minimally invasive spine procedures may be found in U.S. Pat. No. 4,573,448, U.S. Pat. No. 6,217,5009, and U.S. Pat. No. 7,273,468, which are hereby incorporated by reference in their entirety.

In one particular embodiment, a patient is placed into a prone position with a pillow or other structure below the abdomen to limit lumbar lordosis. The patient is prepped and draped in the usual sterile fashion and anesthesia is achieved using general, regional or local anesthesia. Under fluoroscopic guidance, a spinal needle with a stylet is inserted into laterally down to the facet joint adjacent the target foramen location, in generally the same frontal plane as the facet joint, of the patient's back. The spinal needle is then tapped into the facet joint and the stylet is removed. A threaded k-wire is inserted into the spinal needle and then rotated to anchor the K-wire into the facet joint bone. The spinal needle is then removed and a dilator is passed over the K-wire and down to the facet joint. From here, a cannula may be then exchanged with the dilator, or the cannula may be passed into or over the dilator and then the dilator is removed. An endoscopic system is then inserted into the cannula to confirm the target foramen location. The burr or grinder system may then be inserted into the endoscopic system to remove any calcifications or hypertrophic bone at the target foramen location. The burr or grinder system is then positioned against the target tissue using translational, rotational movement, and/or angular movement. The burr or grinder system is then actuated to initiate removal of the calcification or bone, using further translational, rotational movement, and/or angular movement to remove the desired material. In some embodiments, the burr system may be used with an introducer or cannula having an outer diameter of about 0.01 cm to about 1.5 cm or more, sometimes about 0.1 cm to about 1 cm, and other times about 2 mm to about 6 mm.

In alternate embodiments, an anterior procedure through the abdominal cavity or anterior neck region may be performed. In some embodiments where the patient is under local or regional anesthesia, the suspected nerve impingement may be confirmed by contacting or manipulating the suspected nerve with the endoscope, or other instrument inserted through the endoscope, and assessing the patient's response or symptoms.

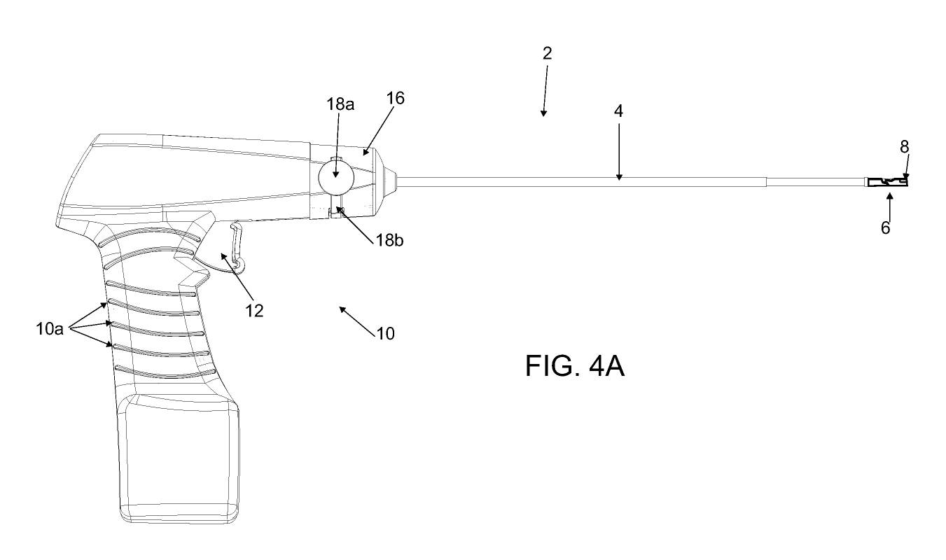

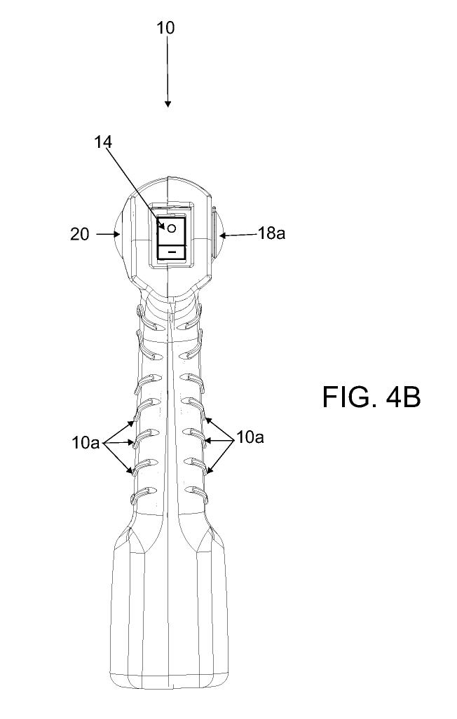

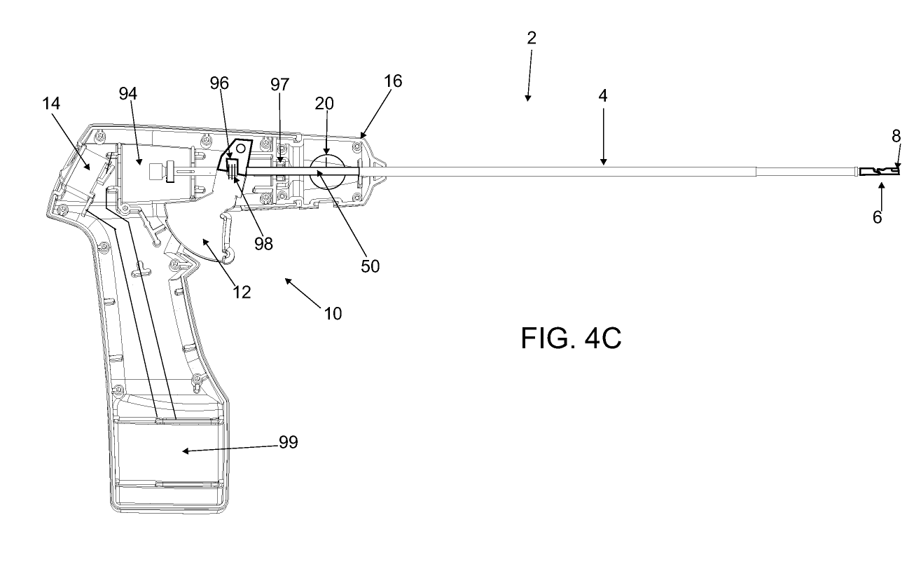

FIGS. 4A and 4B depicts one example of a burr system 2, comprising an outer shaft 4 coupled distally to a burr housing 6 that comprises a rotatable burr element 8. The shaft 4 may be coupled proximally to a proximal housing or handle housing 10. The handle housing 10 may include an adjustment actuator 12 and a power actuator 14. The adjustment actuator 12 is configured to selectively bend or pivot the burr housing 6 and is described in greater detail below. The power actuator 14 depicted in FIG. 4B comprises an on/off switch that turns the system 2 on and off. In other examples, a speed actuator may be provided, such as a slider or dial to adjust the rotational speed of the system 2. In some further examples, the power actuator and the speed actuator may be incorporated together, e.g. where the “off” position comprises a rotational speed of zero. In some further examples, the speed actuator also permits rotational speed in the opposite direction, which may facilitate unraveling of any material caught by the rotational mechanism. In still other examples, the system 2 may be turned on upon initial activation of the adjustment actuator 12, while further activation or manipulation of the adjustment actuator 12 will selectively bend or pivot the burr housing 10.

The handle housing 10 in FIG. 4A may further comprise a trap cavity 16 configured to retain any fluid or particulate material transported from the burr housing 6. The trap cavity 16 may further comprise a cap 18a to permit sampling or removal of any materials therein. The cap 18a may further comprise a tether 18b attached to the trap cavity 16 to avoid inadvertent loss of the cap 18a. In some further variations, the trap cavity 16 may comprise an optically transparent material to facilitate viewing of its contents, and may further comprise a lens element 20, depicted in FIG. 4B, located in a sidewall of the trap cavity 16 that permits magnified viewing of the cavity materials. The handle housing 10 may further comprise one or more ridges 10a, recesses or sections of textured or frictional surfaces, including but not limited to styrenic block copolymers or other polymer surfaces.

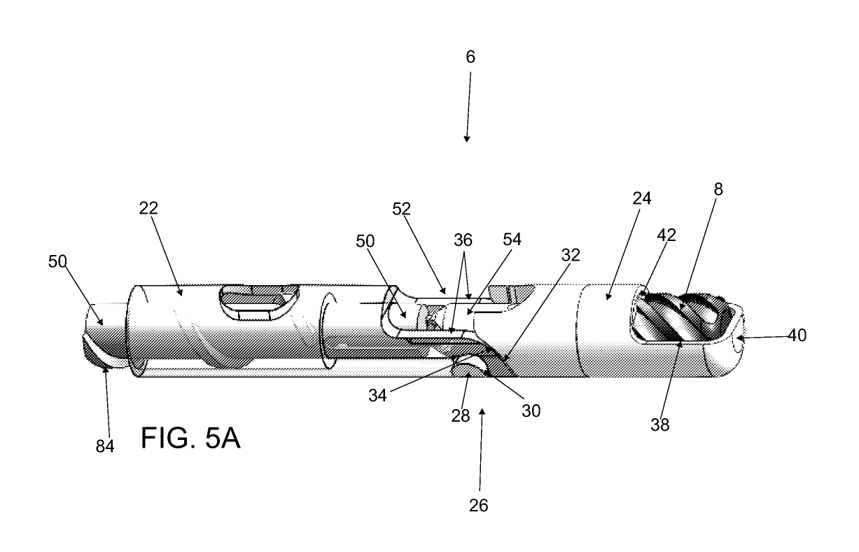

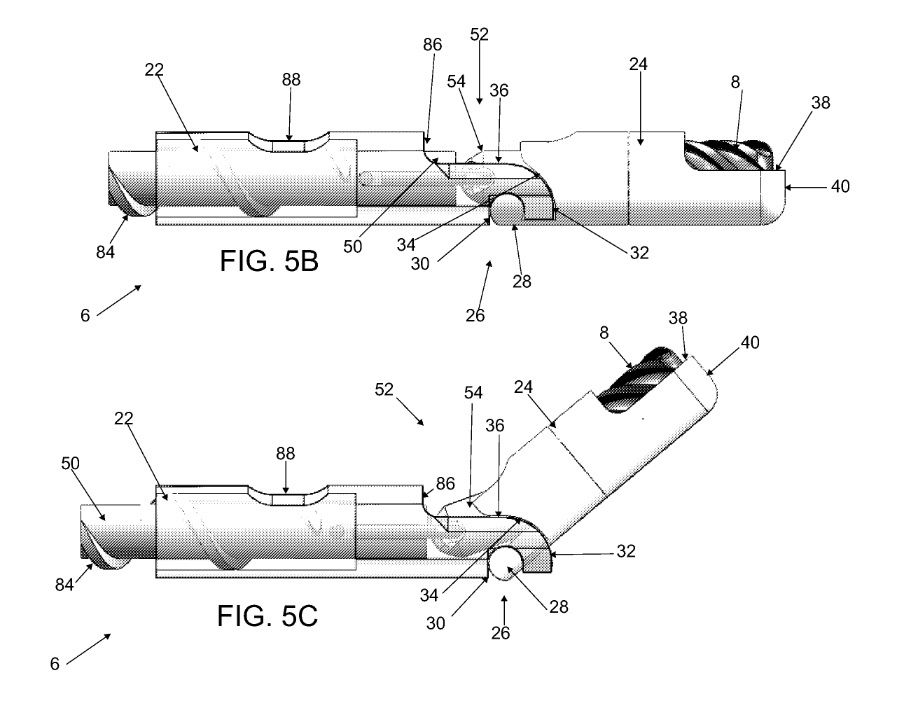

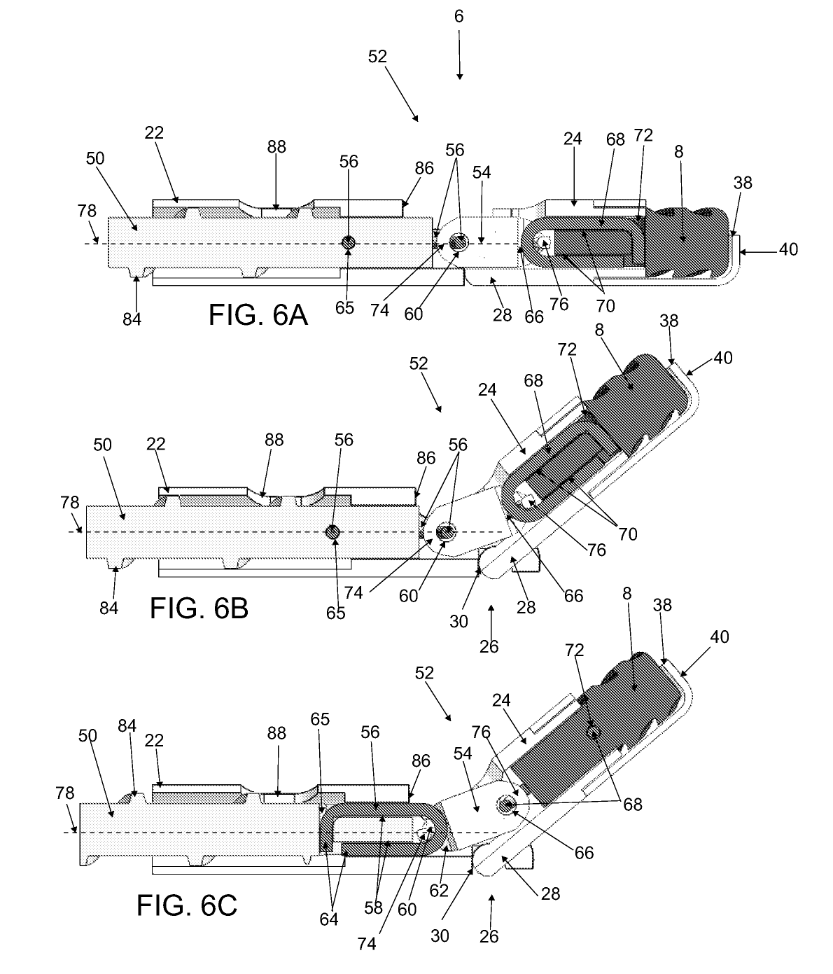

Referring to FIGS. 5A to 6C, the burr housing 6 comprises a proximal housing 22 and a distal housing 24 that are movably coupled together by an articulation assembly 26. In the example depicted in FIGS. 5A to 6C, the articulation assembly 26 comprises a pivot joint provided by a pair of pivot arms 28 projecting proximally and radially outwardly into complementary pivot apertures 30 located on the proximal housing 22. The pivot arms 28 and pivot aperture 30 form a pivot axis of rotation. In some variations, the distance between the pivot axis and the longitudinal axis of the drive shaft 50 may be in the range of about 0.1 mm to about 7 mm or more, in other variations in the range of about 1 mm to about 4 mm, and in still other variations may be in the range of about 0.5 mm to about 2 mm. In other examples, the locations of the pivot arms and apertures may be reversed, or a pivot pin may be provided that couples the proximal and distal housings together. In still other examples, the burr housing may comprise additional housing segments to permit articulation or pivoting at two or more locations.

To further facilitate relative movement between the proximal and distal housings 22 and 24, their corresponding distal and proximal ends 32 and 34 may be configured with complementary shapes. As illustrated in FIGS. 5B and 5C, the distal end 32 of the proximal housing may comprise a convex contour that facilitates sliding of a complementary concave contour of the proximal end 34 of the distal housing 22. The distal end 32 of the proximal housing 22 may also comprise a blocking surface 36 to restrict or limit the range of movement of the distal housing 24.

As shown in FIGS. 5A to 5C, the burr element 8 partially protrudes from a burr opening 38 so that tissue or material to be removed from the body need not bulge or otherwise protrude into the burr opening 38 for removal. The opening 38 may be configured to span at least 25% of the circumference of the distal housing 24, sometimes at least 40% of the distal housing 24, and other times at least 50% of the distal housing 24. To facilitate tissue removal using the distal tip of the burr element 8, the burr opening may be further configured to include a portion of the distal wall 40 or distal tip of the distal housing 24. The burr cavity 42 of the distal housing 24 may be configured to be slightly larger than the burr element 8 to reduce heat generation between the distal housing 24 and the burr element 8 during rotation.

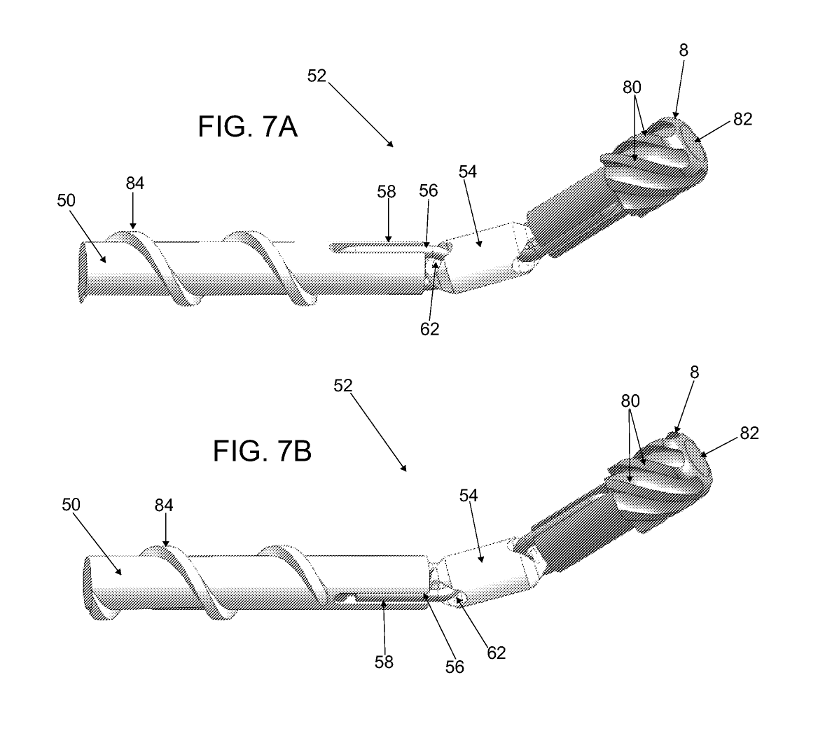

To rotate the burr element 8, the drive shaft 50 is rotated using a motor located within the handle housing 10. Referring to FIGS. 6A to 7B, to facilitate rotation when the distal housing 24 is bent or pivoted relative to the proximal housing 22, the drive shaft 50 may be attached to the burr element 8 with a coupling assembly 52. The coupling assembly 52 is configured to permit angulation between of the burr element 8 relative to the drive shaft 50 while maintaining transmission of rotational forces. The coupling assembly 52 comprises a coupling joint 54 that is pivotably attached to the drive shaft 50 with a proximal clip or loop element 56. The proximal loop element 56 is located in a pair of loop recesses 58 on the outer surface of the drive shaft 50 (best illustrated in FIGS. 6C, 7A and 7B), and in a proximal eyelet or coupling lumen 60 of the coupling joint 54 (best illustrated in FIGS. 6A to 6C). The openings 62 of the coupling lumen 60 may be contoured with a flared configuration to redistribute torsional forces across a length of the proximal loop element 56, rather than concentrating the forces at the edge of the openings. As depicted in FIG. 6C, the proximal loop element 56 may comprise an open loop with two ends 64 located in a drive shaft lumen 65, but in other examples, the ends of the loop element may be fused or integrally formed. In other variations, the ends 64 may be located within the loop lumen 60 of the coupling joint 54, and in still other variations, the loop element may be integrally formed with either the drive shaft or the coupling joint 54. In still other variations, the projecting arms may be used instead of a loop element.

The coupling joint 54 may be further comprise a distal coupling lumen 66 that is coupled to a distal clip or loop element 68 that is attached to distal loop recesses 70 and/or a distal loop lumen 72 located on the burr element 8. The configuration and/or variations of the distal loop element 68 may be similar to that of the proximal loop element 56, or may be different. In further variations, a coupling joint is not used, and a loop element may be used to couple the drive shaft directly to the burr element.

As shown in FIGS. 5B and 5C, to actuate the bending or pivoting of the distal housing 24 relative to the proximal housing 22, the drive shaft 50 is pulled proximally, which then pulls the proximal end 74 of the coupling joint 54 proximally while causing deflection of the distal end 76 of the coupling joint 54 away from the central axis 78 of the drive shaft 50. In some embodiments, the drive shaft 50 may be configured to move longitudinally a length of about 0.01 cm to about 2 cm or more, sometimes about 0.02 cm to about 1.5 cm and other times about 0.05 to about 1 cm.

As illustrated in FIGS. 7A and 7B, the burr element 8 may comprise a plurality of helical cutting ridges 80. Although referenced as a “burr”, the action of the burr element 8 may include cutting, chopping, grinding, debriding, debulking and/or emulsifying tissue. Emulsification includes, for example, forming a suspension of tissue particles in a medium. The medium may comprise existing liquid at the target site, liquid added through the burr system, and/or liquid generated by the debulking of the tissue. The particular angles of the leading edge and trailing edge of each ridge 80 may be the same or different. In the example depicted in FIGS. 7A and 7B, the ridges 80 are limited to the side surface of the burr element 8, but in other examples, the ridges may also extend to the distal surface 82 of the burr element 8.

To facilitate removal of fluid and/or particulate matter generated by the burr element 8 from the target location, an optional port may be provided on the handle housing 10 for attachment of an aspiration or suction source. An aspiration or suction source may be used, for example, to transport fluid or material between the space located between the outer shaft 4 and the drive shaft 50. In some variations, aspiration or suction of material may be provided through the trap cavity 16 by removal of the cap 18a and the attachment of the suction or vacuum apparatus.

As illustrated in FIGS. 5A to 7B, transport of fluid and/or particulate matter may alternately be provided by a helical member or auger 84 located on the surface of the drive shaft 50. To facilitate transport during activation of the burr element 8, the rotational configuration of the auger 84 may matched to the directionality, if any, of the helical ridges 80 of the burr element 8. When rotated in the opposite direction, the auger 84 may be used expel or distally transport tissue, fluid or other materials or agents from the outer shaft 4 or supplied to the trap cavity 16. The burr system 2 may be configured to permit entry of fluid and/or particulate material into the outer shaft 4 through the distal opening 86 of the proximal housing 22 and/or a side opening 88 of the proximal housing 22. In some examples, a cutting edge may be provided at either or both openings 80 and 82, which may facilitate further shearing or break-up of larger tissue fragments or materials.

In some embodiments, the auger 84 may have a longitudinal dimension of about 2 mm to about 10 cm or more, sometimes about 3 mm to about 6 cm, and other times about 4 mm to about 1 cm. In other embodiments, the longitudinal dimension of the auger 84 may be characterized as a percentage of the longitudinal dimension of the outer shaft 4, and may range from about 5% to about 100% of the longitudinal dimension of outer shaft 4, sometimes about 10% to about 50% or more, and other times about 15% to about 25%, and still other times is about 5% to about 15%. Although the auger 84 depicted in FIGS. 5A to 7B will rotate with a tissue debulking element due to its mounting onto drive shaft 50, in other embodiments, the auger 84 may also be configured to rotate independently from drive shaft 50. For example, the auger 84 may comprise a helical coil that is not surface mounted on the drive shaft 50. In still other embodiments, the auger may be mounted on the inner surface of the outer shaft 4 and can be used to transport fluid or material by rotation of the outer shaft 4, independent of the auger or a burr element.

Although the auger 84 is depicted as a continuous structure, in some embodiments, the auger 84 may be interrupted at one or more locations. Also, the degree or angle of tightness of the auger 84 may vary, from about 0.5 turns/mm to about 2 turns/mm, sometimes about 0.75 turns/mm to about 1.5 turns/mm, and other times about 1 turn/mm to about 1.3 turns/mm. The cross-sectional shape of the auger 84 may be generally rounded as depicted in FIGS. 5A to 7B, but in other embodiments, may have one or more edges. The general cross-sectional shape of the auger 84 may be circular, elliptical, triangular, trapezoidal, squared, rectangular or any other shape. The turn tightness and cross-sectional shape or area of the auger 84 may be uniform or may vary along its length. In some embodiments, multiple augers may be provided in parallel or serially within the outer shaft 4.

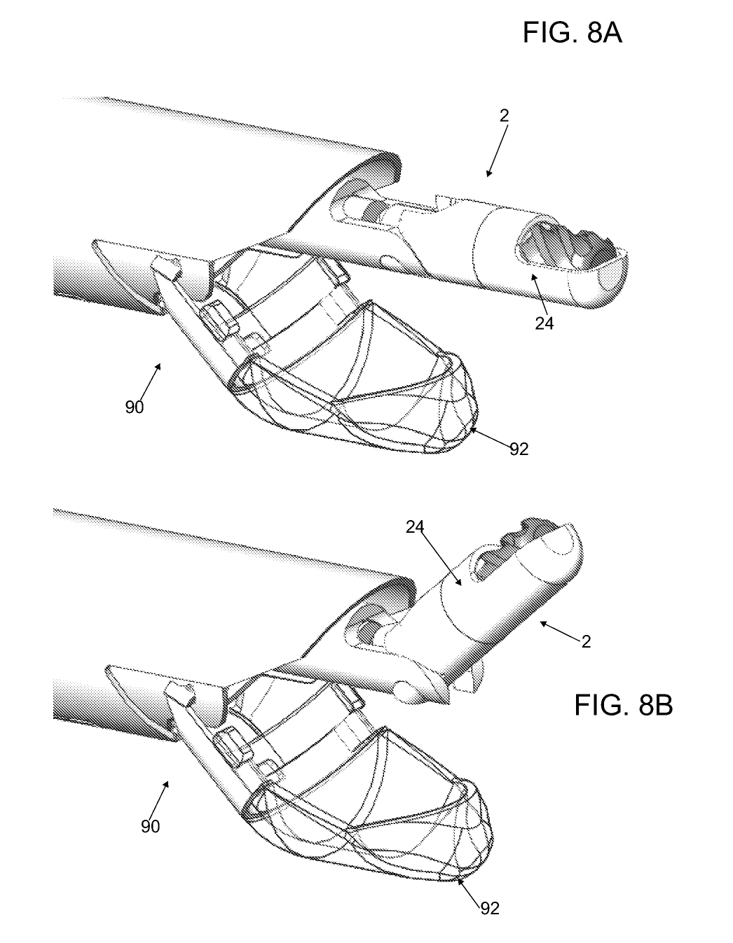

In some embodiments, a protective sheath, barrier or device may be inserted between the nerve and the stenotic structure(s) to protect the nerve during bone removal. The protection device may be a separate device, or may be a component integral with the endoscope or with the bone removal tool, for example. In one example, a flexible cannula tip surrounded by a balloon is used to navigate the anatomical structure of the vertebrae and simultaneously form spacing between tissue and bone in an atraumatic manner to adjust corrective spacing and initially relieve pressure from the bone. U.S. application Ser. No. 11/373,848, which is hereby incorporated by reference in its entirety, discloses a number of embodiments for an endoscopy system comprising an atraumatic tip which may be safely used to displace sensitive or critical soft tissue structures during any of a variety of endoscopic procedures. In another example, U.S. application Ser. No. 11/362,431, which is hereby incorporated by reference in its entirety, discloses an endoscopy system comprising an extendable and steerable balloon device that may be used to manipulate tissues. Once these targeted bone areas are accessed, and nerve structure is displaced, a burr device can be inserted into a channel of the cannula and applied to cut away segments of bone. In some further embodiments, regions of bone hypertrophy or ligament calcification or hardening may be removed using a differential tissue debulking apparatus which preferentially removes certain types of materials while avoiding or reducing damage to other types of tissues. In some embodiments, the differential tissue debulking apparatus may preferentially destroy or debulk soft tissue over hard tissue, but in other embodiments, the differential tissue debulking apparatus may preferentially destroy or debulk hard tissue over soft tissue. The differential tissue debulking apparatus may be an energy transmission device or a mechanical device. In still other examples, such as those depicted in U.S. application Ser. No. 12/582,638, which is hereby incorporated by reference in its entirety, the endoscopic system may comprise one or move movable retractor elements that may be inserted between the target tissue and an adjacent nerve to protect the nerve from damage during the foraminotomy procedure. As shown in FIGS. 8A and 8B, the burr system 2 may be positioned relative to an endoscopic retractor system 90 with an outwardly deflecting retractor element 92 such that the distal housing 24 of the burr system 2 bends or pivots in the generally opposite direction of the retractor element 92. Example of In use, the retractor element 92 may be positioned between the burr element 8 and bony structure to provide leverage as the burr element 8 is applied to target tissue, or between the burr element 8 and an adjacent neural structure to reduce the risk of injury by the burr element 8. The retractor element 92 and distal housing 24, however, may also be positioned with their bending directions at a variety of other relative positions besides 180 degrees apart, including but not limited to about 30 degrees, about 45 degrees, about 60 degrees, about 75 degrees, about 90 degrees, about 105 degrees, about 120 degrees, about 135 degrees, about 150 degrees, about 165 degrees or more, for example.

In other examples, the foraminotomy or foraminoplasty procedures may be performed without any specific protective structure or component for manipulating neural tissue away from the treatment site. In these and other embodiments, precise maneuverability may be a beneficial characteristic for performing a minimally invasive spinal surgery, to permit precise removal of smaller bone sections that are applying pressure on a nerve. For example, the differential tissue debulking apparatus may comprise a rotatable device with a surface configuration that removes bone or other calcified or hardened tissues while generally resisting engagement or removal of softer tissues such as nerves or blood vessels. In one embodiment, the principle underlying a differential tissue debulking apparatus may be demonstrated by assessing the elastic modulus of a material.

Thus, a softer tissue will generally have a lower elastic modulus and therefore more likely to deflect away from the uneven abrading surface of the debulking apparatus rather than engage, and therefore is less likely to be abraded or damaged. The modulus of bone or hardened ligament found in spinal stenosis tissue is typically up to about 4 to about 5 orders of magnitudes higher than that of nerves and blood vessels. At a finer burr roughness, the nerves, blood vessels and other soft tissue will atraumatically deform with respect to such a debulking apparatus and not be damaged, while harder stenotic tissue will resist deformation and are impacted and damaged.

To configure a rotatable burr or cutting device, for example, to exert a particular relative tangential force, the density or spacing between the abrasive or cutting structures may be altered. In some embodiments, by increasing the density or decreasing the spacing of the tissue removal structures, the frictional or engagement force between the tissue removal element and the tissue is distributed among a greater number of structures and less concentrated. A broader distribution of force may permit soft tissues to deform in response to a rotating burr or cutting device and thereby avoid significant damage, while bone or calcified tissues are unable to substantially deform and will be abraded or removed. In some embodiments where the differential tissue removal apparatus comprises a rotatable burr, the burr may have a roughness of about 50 grit to about 1000 grit or more, sometimes in the range of about 100 grit to about 500 grit, and other times about 120 to 200. Alternatively, the roughness of the burr can be expressed in grit size as well as particle spacing. In some embodiments, grit size may be in the range of about 0.0005 inches to 0.01 inches or more, or sometimes in the range of about 0.001″ to about 0.01″, and other times in the range of about 0.001 inches to 0.004 inches. Also, the angle of the abrasive or cutting structures with respect to the device surface may also be configured from about 0 degrees to about 180 degrees, sometimes about 45 degrees to about 90 degrees, and other times about 70 degrees to about 90 degrees. In some embodiments, burr devices with finer grits may be used generate greater heat at the target site and may exhibit greater hemostasis function than burr devices with coarser grits.

In one example, depicted in FIGS. 8A to 8D, the differential tissue removal apparatus comprises a burr element 200 with a plurality of abrasive structures 202 located on a tissue removal section 204. The burr element 200 further comprises a distal tip 206 and proximal shaft 208, but in other embodiments, the burr element 200 may comprise a distal shaft instead of a distal tip 206. The burr element 200 has a generally cylindrical shape, but in other embodiments, the burr element may be elliptical, conical, or any of a variety of other shapes. The cross-sectional shape of the burr element may be circular, ovoid, triangular, squared, rectangular or any other shape, and need not be the same along the longitudinal length of the burr element 200. As depicted in FIG. 8A, the distal tip 206 of the may have a generally convex shape, but in other embodiments, the distal tip may be generally concave, tapered, or flat, for example. The distal tip 206 may have a smooth surface, or may be covered with cutting or abrasive structures.

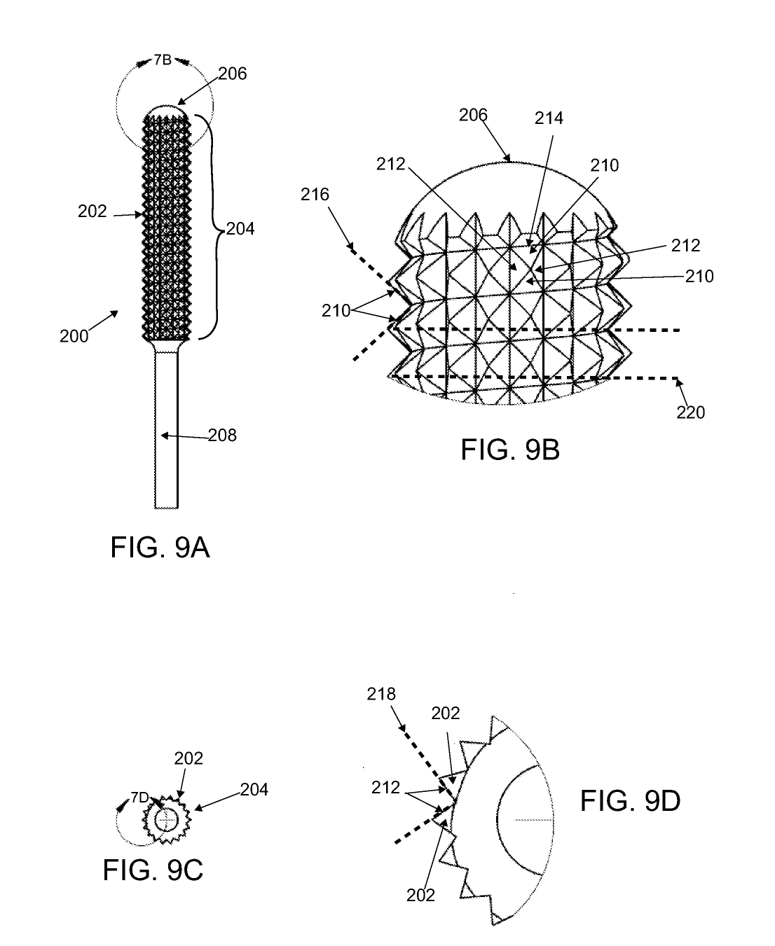

The abrasive structures 202, seen best in FIG. 9B, may comprise a four-sided pyramidal shape with a square base. The sides 210 and 212 of the abrasive structure 202 have a generally triangular shape with a base 214 that contacts the bases 214 of the adjacent abrasive structures 202. In other embodiments, the bases 214 of the abrasive structures 202 may be spaced apart longitudinally and/or circumferentially about 0.001 inches to about 0.06 inches, and other times about 0.006 inches to about 0.03 inches. As shown in FIGS. 9B and 9D, the angle 216 between two adjacent sides 210 of two longitudinally adjacent abrasive structures 202 is about 90 degrees, and the angle 218 between two adjacent sides 212 of two circumferentially adjacent abrasive structures 202 is about 90 degrees. In other embodiments, however, the inter-structures angles 216, 218 may be different, and may range from about 25 degrees to about 165 degrees, sometimes about 45 degrees to about 135 degrees, and other times about 65 degrees to about 100 degrees. Although the sides 210 and 212 of the abrasive structures 202 in FIG. 9B have planar configurations, in other embodiments, one or more sides may be convex, concave or other type of non-planar configuration. In some embodiments, the abrasive structures 202 may be aligned with adjacent abrasive structures or may be offset. For example, the abrasive structures 202 depicted in FIG. 9B have a pitch of about 0.012 inches, or about 200% of the longitudinal length of one abrasive structure 202. In other embodiments, the abrasive structures may have a pitch in the range of about 0.001 inches to about 0.06 inches, and other times about 0.006 inches to about 0.03 inches. Relative the longitudinal length of the abrasive structure, the abrasive structures may have a pitch in the range of about 5% to about 500% or more, sometimes about 50% to about 300%, and other times about 100% to about 200%. The abrasive structures 202 need not have a uniform size, shape, orientation or spacing.

The abrasive structures may comprise any of a variety of other shapes, including but not limited to a three-sided pyramid, a frusto-pyramidal shape, a conical or frusto-conical shape, or any other type of tapered shape. In other examples, the abrasive structures may comprise a square or rectangular block configuration, or any other type of polygonal block configuration. Alternatively, the abrasive structures may comprise one or more ridge or edge structures, which may comprise one or more curves or angles. Although the abrasive structures 202 depicted in FIG. 9B have main axes between their bases and distal tips that are generally centered about their bases, in other embodiments, the main axes may be eccentrically located. The main axes may also be perpendicular, or acutely or obtusely angled with respect to the bases. In some embodiments, the main axes of the abrasive structures may have an angle with respect to the base of the abrasive structures that is in the range of about 5 degrees to about 175 degrees, or in the range of about 45 degrees to about 135 degrees, or in the range of about 25 degrees to about 90 degrees. In some embodiments, the main axis of the abrasive structures may be characterized with respect to the direction of motion when the tissue debulking apparatus is rotated. In some embodiments, the tip or edge of the debulking structure may be characterized as having a negative, zero, or positive rake angle. In some embodiments, providing the abrasive or cutting structures with a negative rake angle (e.g. angled away from the direction of motion) may reduce the abrasive or cutting torque of the device but may increase the differential cutting characteristic of the device. In some embodiments, the device may be bi-directional and have abrasive or cutting structures configured with different rake angles in each direction, e.g. a negative rake angle in one direction and a positive rake angle in the other direction.

The length of the tissue removal section 204 of the burr element 200 may be in the range of about 0.1 inches to about 0.5 inches, examples, may be in the range of 0.2 inches to about 0.3 inches, and in still other examples, may be in the range of about 0.25 inches to about 0.75 inches. The tissue removal section 204 may have a diameter or maximum transverse width in the range of about 0.01 inches to about 0.1 inches, about 0.02 inches to about 0.08 inches, or about 0.4 inches to about 0.6 inches.

The burr element 8 or 200 may comprise any of a variety of one or more materials, including but not limited to nickel-titanium alloy, stainless steel, cobalt-chromium alloy, nickel-cobalt-chromium-molybdenum alloy, titanium-aluminum-vanadium alloy, tungsten carbide, silica carbide, diamond, and ceramic. The abrasive structures 202 may comprise the same material as the rest of the burr element 200 or may comprise a different material. In some embodiments, the abrasive structures 202 may comprise a harder material, such as diamond, glass, quartz, tungsten carbide, cobalt chromium, and ceramics.

Referring to FIG. 4C, the drive shaft 50 extends proximally out of the outer shaft 4 and through the trap cavity 16 and is rotatably coupled to a motor 94 while permitting longitudinal displacement. For example, longitudinal outer ridges located on the drive shaft may interface with longitudinal inner ridges located in a coupling lumen of the motor to rotatably couple the drive shaft to the motor while permitting longitudinal sliding between the shaft and motor. Of course, in other examples, the relationship between the outer and inner ridges may be reversed with respect to the drive shaft and motor, or a different type of rotatably coupled but longitudinally movable linkage may be provided. To facilitate longitudinal movement of the drive shaft 50, the adjustment actuator 12 may be configured with a recess or opening 96 (or projection) that is configured to exert axial pulling and pushing forces against a flange 98 or other rotating structure on the outer surface of the drive shaft 50.

In some examples, the motor 94 of the burr system 2 is a DC motor, but in other embodiments, the motor 94 may be configured with any of a variety of motors, including but not limited to an AC or a universal motor. The motor 94 may be a torque, brushed, brushless or coreless type of motor. In some embodiments, the motor 94 may be configured to provide a rotational speed of about 500 rpm to about 200,000 rpm, sometimes about 1,000 rpm to about 40,000 rpm, and at other times about 5,000 rpm to about 20,000 rpm. The motor 94 may act on the burr element 8 via the outer shaft 4, or a by drive member located within the outer shaft 4. A fluid seal 97 may be provided to protect the motor 94 and/or other components of the handle housing 10 from any fluids or other materials that may be transported through the outer shaft 4 or from the trap cavity 16. The power to the motor 94 is controlled by the power actuator 14 and is powered by the battery 99.

It is to be understood that this invention is not limited to particular exemplary embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a blade” includes a plurality of such blades and reference to “the energy source” includes reference to one or more sources of energy and equivalents thereof known to those skilled in the art, and so forth.

The publications discussed herein are provided solely for their disclosure. Nothing herein is to be construed as an admission that the present invention is not entitled to antedate such publication by virtue of prior invention. Further, the dates of publication provided, if any, may be different from the actual publication dates which may need to be independently confirmed.

What is claimed is:

1. A burr system, comprising:
a proximal handle with a trap cavity;
an outer shaft attached to the proximal handle and movably coupled to a deflectable burr housing;
a rotatable burr located in the burr housing and protruding from a burr opening located along a side wall and a distal end of the distal housing;
a drive shaft comprising a rotational axis and configured to be axially displaceable and rotatably coupled to a motor located in the proximal handle; and
a coupling element pivotably coupled to the threaded drive shaft and the rotatable burr.

2. The burr system of claim 1, wherein the coupling element is pivotably coupled to the threaded drive shaft and the rotatable burr with a wire clip.

3. The burr system of claim 1, wherein the outer shaft is movably coupled to the deflectable burr housing with a pivot joint.

4. The burr system of claim 3, wherein the pivot joint comprises a pivot axis that is located about 0.1 mm to about 7 mm from the rotational axis of the drive shaft.

5. The burr system of claim 1, wherein the coupling element is configured with a variable rotation axis with respect to the rotational axis of the drive shaft.

6. The burr system of claim 5, wherein the variable rotation axis of the coupling element has a variable angle with respect to the rotational axis of the drive shaft in the range of about 0 degrees to about 90 degrees.

7. The burr system of claim 1, wherein the coupling element is configured with a variable rotation axis with respect to the rotational axis of the rotatable burr.

8. The burr system of claim 7, wherein the variable rotation axis of the coupling element has a variable angle with respect to the rotational axis of the rotatable burr in the range of about 0 degrees to about 90 degrees.

9. The burr system of claim 1, wherein the coupling element is configured to transmit torque from the drive shaft to the rotatable burr while free to rotate about variable rotational axis relative to the rotational axis of the drive shaft.

10. The burr system of claim 1, wherein the coupling element is linked to the drive shaft and the rotatable burr by a proximal arcuate member and a distal arcuate member.

11. The burr system of claim 10, wherein:
the proximal arcuate member is fixedly attached to the drive shaft in a first plane; and
the distal arcuate member if fixedly attached to the coupling element in a second plane that is generally transverse to the first plane.

12. The burr system of claim 11, wherein the first plane and the second plane maintain a generally transverse relationship during rotation of the coupling element.

13. The burr system of claim 1, where the drive shaft is a threaded drive shaft.

14. A method of treating a patient, comprising:
inserting a shaft of a burr system into a patient;
removing a first calcified material using a burr of the burr system;
deflecting the burr relative to the shaft; and
removing a second calcified material using the deflected burr.

15. The method of claim 14, further comprising:
passing the burr system through an endoscopic retractor; and
positioning a retractor element of the endoscopic retractor between the first calcified material and an adjacent neural structure.
